(19) United States
(12) Patent Application Publication
(10) Pub. No.: US 2004/0166502 A1
(43) Pub. Date: Aug. 26, 2004

(54) Microelectromagnetic dispenser heads and uses thereof

(76) Inventors: Yaming Lai, Jiangxi (CN); Yuxiang Zhou, Beijing (CN); Zhiming Li, Fujian (CN); Junquan Xu, San Diego, CA (US); Litian Liu, Beijing (CN)

Correspondence Address: MORRISON & FOERSTER LLP, 3811 VALLEY CENTRE DRIVE, SUITE 500, SAN DIEGO, CA, 92130-2332, US

(21) Appl. No.: 10/469,318

(22) PCT Filed: Mar. 12, 2002
(86) PCT No.: PCT/US02/07464

(30) Foreign Application Priority Data
Mar. 13, 2001 (CN) 01 1 04398.9

(51) Int. Cl. C12Q 1/68; G01N 33/553

(52) U.S. Cl. 435/6; 436/526

(57) ABSTRACT

This invention relates generally to the field of moiety or molecule transfer. In particular, the invention provides a microelectromagnetic dispenser head, which head comprises: a core comprising a magnetizable substance, said core surrounded by a microcoil suitable for transmitting electrical current and generating a magnetic field via said magnetizable substance and said core having a tip suitable for attracting a magnetic or magnetically labeled moiety; and preferably further comprising one or both of the following: i) a shell that substantially shields magnetic field, generated via said microcoil, from the non-tip portion of said core; and/or ii) a cooling means for cooling said tip. Microelectromagnetic dispensers comprising the heads and methods for transferring moieties using the heads and the microelectromagnetic dispensers are also provided.

The present application claims priority benefit of Chinese Patent Application Serial No. 01104398.9, filed Mar. 13, 2001. The content of the above Chinese Patent Application is incorporated by reference herein in its entirety.

1. Technical Field

This invention relates generally to the field of moiety or molecule transfer. In particular, the invention provides a microelectromagnetic dispenser head, which head comprises: a core comprising a magnetizable substance, said core surrounded by a microcoil suitable for transmitting electrical current and generating a magnetic field via said magnetizable substance and said core having a tip suitable for attracting a magnetic or magnetically labeled moiety; and preferably further comprising one or both of the following: i) a shell that substantially shields magnetic field, generated via said microcoil, from the non-tip portion of said core; and/or ii) a cooling means for cooling said tip. Microelectromagnetic dispensers comprising the heads and methods for transferring moieties using the heads and the microelectromagnetic dispensers are also provided.

2. Background Art

In many fields such as biology, medicine and chemistry fields, many approaches are used to transport and dispense liquid or solid samples, which may include many different types of moieties, such as proteins, nucleic acids, viruses, cells, cellular organelles, and tissues or complex thereof. For large volume samples, it is easy to transport and dispense. But many experiments require manipulation and dispensation of small volume samples. For example, in many experimental protocols for molecular biology work, the precision pipettes are used to manipulate micro- or sub-micro-liter sample. And for most manipulation devices, the sub-micro-liter sample is the limit. In order to avoid contamination, the devices either can be used only once or need special cleaning. All of these are expensive, inconvenient and time consuming.

The development of biochip technology needs more advanced sample transportation and dispensation technology. The sample volume is small and the sample class is numerous. For example, in microarray technology, many kinds of probes are immobilized on the surface of different substrates, e.g., glass, silicon, nylon membrane. The probes can be DNA, RNA, protein, cell or tissue, which include specific biological information. Microdispensers are commonly used to dispense the liquid sample to different locations of the microarray and these microdispensers are controlled by precision robotics, which can manipulate and locate the microdispensers. The volume of the liquid sample dispensed by microdispensers is around several hundred picoliter or several nanoliter. For high throughput use, the probe numbers in one microarray can be several thousand to several hundred thousand. Thus, suitable dispense technology is required.

The precision robotics are used in modem industry for many years. The limitation for the dispense technology lies in microdispensers. At present, the commonly used dispensers include solid pin structures, capillaries, piezoelectric print heads, etc. Most of them are very expensive and hence not disposable. Special cleaning technology must be used to avoid contamination. Sample can be wasted during these procedures. This will decrease the efficiency of the dispense procedures and the contamination may still occur.

Accordingly, how to transport and dispense different kinds of samples quickly and efficiently is an important problem for biochip technology. This invention address this and other related needs in the art.

### Disclosure of the Invention

In one aspect, the present invention is directed to a microelectromagnetic dispenser head, which head comprises: a) a core comprising a magnetizable substance, said core surrounded by a microcoil suitable for transmitting electrical current and generating a magnetic field via said magnetizable substance and said core having a tip suitable for attracting a magnetic or magnetically labeled moiety. Preferably, the microelectromagnetic dispenser head can further comprise one or both of the following: i), a shell that substantially shields magnetic field, generated via said microcoil, from the non-tip portion of said core; and/or ii) a cooling means for cooling said tip. Microelectromagnetic dispensers comprising the heads or arrays of the heads are also provided.

In another aspect, the present invention is directed to a method for transferring a moiety, which method comprises: a) providing a magnetic or magnetically labeled moiety to be transferred at first location, b) providing a microelectromagnetic dispenser head comprising a core comprising a magnetizable substance, said core surrounded by a microcoil suitable for transmitting electrical current and generating a magnetic field via said magnetizable substance and said core having a tip suitable for attracting said magnetic or magnetically labeled moiety; c) attracting said magnetic or magnetically labeled moiety from said first location to said tip of said microelectromagnetic dispenser head; and d) transferring and releasing said magnetic or magnetically labeled moiety to a second location from said tip of said microelectromagnetic dispenser head.

### BRIEF DESCRIPTION OF THE DRAWINGS

FIG. 1 illustrates an exemplary microelectromagnetic dispenser head.

FIG. 2 illustrates an exemplary microelectromagnetic dispenser head with a cooling system.

FIG. 3 illustrates an exemplary microelectromagnetic dispenser head containing multiple tips and an exemplary array of microelectromagnetic dispenser heads.

FIG. 4 illustrates a schematic procedure of liquid sample transferring with an exemplary microelectromagnetic dispenser head.

### MODES OF CARRYING OUT THE INVENTION

For clarity of disclosure, and not by way of limitation, the detailed description of the invention is divided into the subsections that follow.

A. Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “magnetic substance” refers to any substance that has the properties of a magnet, pertaining to a magnet or to magnetism, producing, caused by, or operating by means of, magnetism.

As used herein, “magnetizable substance” refers to any substance that has the property of being interacted with the field of a magnet, and hence, when suspended or placed freely in a magnetic field, of inducing magnetization and producing a magnetic moment. Examples of magnetizable substance include, but are not limited to, paramagnetic, ferromagnetic and ferrimagnetic substances.

As used herein, “paramagnetic substance” refers to the substances where the individual atoms, ions or molecules possess a permanent magnetic dipole moment. In the absence of an external magnetic field, the atomic dipoles point in random directions and there is no resultant magnetization of the substances as a whole in any direction. This random orientation is the result of thermal agitation within the substance. When an external magnetic field is applied, the atomic dipoles tend to orient themselves parallel to the field, since this is the state of lower energy than antiparallel position. This gives a net magnetization parallel to the field and a positive contribution to the susceptibility. Further details on “paramagnetic substance” or “paramagnetism” can be found in various literatures, e.g., at Page 169-page 171, Chapter 6, in “Electricity and Magnetism” by B. I Bleaney and B. Bleaney, Oxford, 1975.

As used herein, “ferromagnetic substance” refers to the substances that are distinguished by very large (positive) values of susceptibility, and are dependent on the applied magnetic field strength. In addition, ferromagnetic substances may possess a magnetic moment even in the absence of the applied magnetic field, and the retention of magnetization in zero field is known as “remanence”. Further details on “ferromagnetic substance” or “ferromagnetism” can be found in various literatures, e.g., at Page 171-page 174, Chapter 6, in “Electricity and Magnetism” by B. I Bleaney and B. Bleaney, Oxford, 1975.

As used herein, “ferrimagnetic substance” refers to the substances that show spontaneous magnetization, remanence, and other properties similar to ordinary ferromagnetic materials, but the spontaneous moment does not correspond to the value expected for full parallel alignment of the (magnetic) dipoles in the substance. Further details on “ferrimagnetic substance” or “ferrimagnetism” can be found in various literatures, e.g., at Page 519-524, Chapter 16, in “Electricity and Magnetism” by B. I Bleaney and B. Bleaney, Oxford, 1975.

As used herein, “metal oxide particle” refers to any oxide of a metal in a particle form. Certain metal oxide particles have paramagnetic or super-paramagnetic properties. “Paramagnetic particle” is defined as a particle which is susceptible to the application of external magnetic fields, yet is unable to maintain a permanent magnetic domain. In other words, “paramagnetic particle” may also be defined as a particle that is made from or made of “paramagnetic substances”. Non-limiting examples of paramagnetic particles include certain metal oxide particles, e.g., Fe₃O₄ particles, metal alloy particles, e.g., CoTaZr particles.

As used herein, “chip” refers to a solid substrate with a plurality of one-, two- or three-dimensional micro structures or micro-scale structures on which certain processes, such as physical, chemical, biological, biophysical or biochemical processes, etc., can be carried out. The micro structures or micro-scale structures such as, channels and wells, electrode elements, electromagnetic elements, are incorporated into, fabricated on or otherwise attached to the substrate for facilitating physical, biophysical, biological, biochemical, chemical reactions or processes on the chip. The chip may be thin in one dimension and may have various shapes in other dimensions, for example, a rectangle, a circle, an ellipse, or other irregular shapes. The size of the major surface of chips used in the present invention can vary considerably, e.g., from about 1 mm² to about 0.25 m². Preferably, the size of the chips is from about 4 mm² to about 25 cm² with a characteristic dimension from about 1 mm to about 7.5 cm. The chip surfaces may be flat, or not flat. The chips with non-flat surfaces may include channels or wells fabricated on the surfaces. One example of a chip is a solid substrate onto which multiple types of DNA molecules or protein molecules or cells are immobilized.

As used herein, “medium (or media)” refers to a fluidic carrier, e.g., liquid or gas, wherein a moiety, alone or bound to a magnetic particle, is dissolved, suspended or contained.

As used herein, “microfluidic application” refers to the use of microscale devices, e.g., the characteristic dimension of basic structural elements is in the range between less than 1 micron to 1 cm scale, for manipulation and process in a fluid-based setting, typically for performing specific biological, biochemical or chemical reactions and procedures. The specific areas include, but are not limited to, biochips, i.e., chips for biologically related reactions and processes, chemchips, i.e., chips for chemical reactions, or a combination thereof The characteristic dimensions of the basic elements refer to the single dimension sizes. For example, for the microscale devices having circular shape structures (e.g. round electrode pads), the characteristic dimension refers to the diameter of the round electrodes. For the devices having thin, rectangular lines as basic structures, the characteristic dimensions may refer to the width or length of these lines.

As used herein, “micro-scale structures” means that the structures have characteristic dimension of basic structural elements in the range from about 1 micron to about 20 mm scale.

As used herein, “moiety” refers to any substance whose transfer using the present microelectromagnetic dispenser head is desirable. Normally, the dimension (or the characteristic dimensions) of the moiety should not exceed 1 cm. For example, if the moiety is spherical or approximately spherical, the dimension of the moiety refers to the diameter of the sphere or an approximated sphere for the moiety. If the moiety is cubical or approximately cubical, then the dimension of the moiety refers to the side width of the cube or an approximated cube for the moiety. If the moiety has an irregular shape, the dimension of the moiety may refer to the average between its largest axis and smallest axis. Non-limiting examples of moieties include cells, cellular organelles, viruses, particles, molecules, e.g., proteins, DNAs and RNAs, or an aggregate or complex thereof.

As used herein, “magnetic or magnetically labeled moiety” refers to any moiety that is intrinsically magnetic or is made magnetic by attaching to or associating with a magnetic label. The moiety can be attached to or associated with the magnetic label directly or via a linker. Non-limiting examples of magnetic labels include magnetic beads of different sizes (e.g., from about 0.5 to about 20 microns in diameter).

As used herein, “high permeability material” refers to materials that have high magnetic permeability and can efficiently conduct magnetic lines of force (or magnetic field). The ability to conduct magnetic field is called permeability, and in a magnetic-shield material, the degree of permeability is expressed numerically. The standard or base line is free space with a rating of one, compared with high permeability material having permeability value ranging from about 5,000 to 350,000. Depending on the magnitude of the magnetic field to be shielded, high permeability materials with different permeability values and different magnetic saturation points may be used. For example, to shield a moderate magnetic field of 5,000 gauss, a high permeability material with permeability value of about 150,000 with a saturation point of about 7,500 gauss may be used. In another example, to shield a magnetic field of 12,000 gauss, a high permeability material with permeability value of about 80,000 with a saturation point of about 15,000 gauss may be used.

As used herein, “high thermal conductivity material” refers to materials that have high thermal conductivity and can efficiently conduct heat. Non-limiting examples of high thermal conductivity materials include aluminum, copper, zinc, gold, silver, silicon and tungsten. Preferably, high thermal conductivity materials for cooling purposes have thermal conductivity over 1 J sec⁻¹ cm⁻¹ ° K.⁻¹ at room temperature (about 290-300 ° K.).

As used herein, “a shell that substantially shields magnetic field, generated via said microcoil, from the non-tip portion of said core” means that magnetic field generated from the core is confined at a region that is at close distance from the tip and attract the magnetic or magnetically-labeled moieties within this region to the tip portion, and any non-shielded magnetic field from the non-tip portion of the core, if any, will not result in a strong magnetic field sufficient for attracting the magnetic or magnetically labeled moiety, which is located further away from the tip, to the tip portion. Depending on the application, the magnetic field generated from the core should be confined at regions that are within 100 micron from the tip, i.e., magnetic moiety or magnetically labeled moiety that is located 100 microns away from the tip is not attracted to the tip of the core by the magnetic field. Preferably, the magnetic field generated from the core should be confined at regions that are within 50, 20, 10, 5, 2 microns from the tip. Normally, a shell should shield at least 50% of the magnetic field generated via said microcoil from the non-tip portion of said core. Preferably, a shell should shield at least 60%, 70%, 80%, 90%, 95%, 99%, 99.5%, 99.9% or 100% of the magnetic field generated via said microcoil from the non-tip portion of said core.

As used herein, “optical labeling substance” refers to any optically detectable substance that can be used to label the moiety to be transferred. Quantum dot is an example of an optical labeling substance.

As used herein, “scattered-light detectable particle” refers to any particle that can emit unique and identifiable scattered-light upon illumination with light under appropriate conditions. The nano-sized particles with certain “resonance light scattering (RLS)” properties are examples of one type of “scattered-light detectable particle.”

As used herein, “quantum dot” refers to a fluorescent label comprising water-soluble semiconductor nanocrystal(s). One unique feature of a quantum dot is that its fluorescent spectrum is related to or determined by the diameter of its nanocrystals(s). “Water-soluble” is used herein to mean sufficiently soluble or suspendable in a aqueous-based solution, such as in water or water-based solutions or physiological solutions, including those used in the various fluorescence detection systems as known by those skilled in the art. Generally, quantum dots can be prepared which result in relative monodispersity; e.g., the diameter of the core varying approximately less than 10% between quantum dots in the preparation. Details of quantum dots and how they can be incorporated into microbeads may be found in the literatures, for example, in the articles by Chan and Nie, Science, 281:2016 (1998) and by Han et al., Nature Biotehnology, 19:631-635 (2001).

As used herein, “plant” refers to any of various photosynthetic, eucaryotic multi-cellular organisms of the kingdom Plantae, characteristically producing embryos, containing chloroplasts, having cellulose cell walls and lacking locomotion.

As used herein, “animal” refers to a multi-cellular organism of the kingdom of Animalia, characterized by a capacity for locomotion, nonphotosynthetic metabolism, pronounced response to stimuli, restricted growth and fixed bodily structure. Non-limiting examples of animals include birds such as chickens, vertebrates such fish and mammals such as mice, rats, rabbits, cats, dogs, pigs, cows, ox, sheep, goats, horses, monkeys and other non-human primates.

As used herein, “bacteria” refers to small prokaryotic organisms (linear dimensions of around 1 micron) with non-compartmentalized circular DNA and ribosomes of about 70S. Bacteria protein synthesis differs from that of eukaryotes. Many anti-bacterial antibiotics interfere with bacteria proteins synthesis but do not affect the infected host.

As used herein, “eubacteria” refers to a major subdivision of the bacteria except the archaebacteria. Most Gram-positive bacteria, cyanobacteria, mycoplasmas, enterobacteria, pseudomonas and chloroplasts are eubacteria. The cytoplasmic membrane of eubacteria contains ester-linked lipids; there is peptidoglycan in the cell wall (if present); and no introns have been discovered in eubacteria.

As used herein, “archaebacteria” refers to a major subdivision of the bacteria except the eubacteria. There are three main orders of archaebacteria: extreme halophiles, methanogens and sulphur-dependent extreme thermophiles. Archaebacteria differs from eubacteria in ribosomal structure, the possession (in some case) of introns, and other features including membrane composition.

As used herein, “virus” refers to an obligate intracellular parasite of living but non-cellular nature, consisting of DNA or RNA and a protein coat. Viruses range in diameter from about 20 to about 300 nm. Class I viruses (Baltimore classification) have a double-stranded DNA as their genome; Class II viruses have a single-stranded DNA as their genome; Class III viruses have a double-stranded RNA as their genome; Class IV viruses have a positive single-stranded RNA as their genome, the genome itself acting as mRNA; Class V viruses have a negative single-stranded RNA as their genome used as a template for mRNA synthesis; and Class VI viruses have a positive single-stranded RNA genome but with a DNA intermediate not only in replication but also in mRNA synthesis. The majority of viruses are recognized by the diseases they cause in plants, animals and prokaryotes. Viruses of prokaryotes are known as bacteriophages.

As used herein, “fungus” refers to a division of eucaryotic organisms that grow in irregular masses, without roots, stems, or leaves, and are devoid of chlorophyll or other pigments capable of photosynthesis. Each organism (thallus) is unicellular to filamentous, and possesses branched somatic structures (hyphae) surrounded by cell walls containing glucan or chitin or both, and containing true nuclei.

As used herein, “binding partners” refer to any substances that bind to the moieties with desired affinity or specificity. Non-limiting examples of the binding partners include cells, cellular organelles, viruses, particles, microparticles or an aggregate or complex thereof, or an aggregate or complex of molecules, or specific molecules such as antibodies, single stranded DNAs. The binding partner can be a substance that is coated on the surface of a magnetic particle. Alternatively, the binding partner can be a substance that is incorporated, e.g., microfabricated, into the material composition of a magnetic particle. The material composition of the magnetic particle, in addition being a substrate, may possess binding affinity to certain moiety, and thus functioning as a binding partner itself.

As used herein, “microparticles” refer to particles of any shape, any composition, any complex structures that can be used in the present moiety transferring methods. One example of microparticles is magnetic beads that are manipulatable by magnetic forces. The microparticles used in the methods can have a dimension from about 0.01 micron to about ten centimeters. Preferably, the microparticles used in the methods have a dimension from about 0.01 micron to about several thousand microns.

As used herein, “physical force” refers to any force that moves the moieties or their binding magnetic particles without chemically or biologically reacting with the moieties and the magnetic particles, or with minimal chemical or biological reactions with the magnetic particles and the moieties so that the biologicauchemical functions/properties of the magnetic particles and the moieties are not substantially altered as a result of such reactions. Throughout the application, the term of “forces” or “physical forces” always means the “forces” or “physical forces” exerted on a moiety or moieties, the binding partner(s) and/or the magnetic bead(s). The “forces” or “physical forces” are always generated through “fields” or “physical fields”. The forces exerted on moieties, the binding partner(s) and/or the magnetic bead(s) by the fields depend on the properties of the moieties, the binding partner(s) and/or the magnetic bead(s). Thus, for a given field or physical field to exert physical forces on a moiety, it is necessary for the moiety to have certain properties. While certain types of fields may be able to exert forces on different types of moieties having different properties, other types of fields may be able to exert forces on only limited type of moieties. For example, magnetic field can exert forces or magnetic forces only on magnetic particles or moieties having certain magnetic properties, but not on other particles, e.g., polystyrene microdevices. On the other hand, a non-uniform electric field can exert physical forces on many types of moieties such as polystyrene microdevices, cells, and also magnetic particles. It is not necessary for the physical field to be able to exert forces on different types of moieties or different moieties. But it is necessary for the physical field to be able to exert forces on at least one type of moiety or at least one moiety, the binding partner(s) and/or the magnetic bead(s).

As used here in, “electric forces (or electrical forces)” are the forces exerted on moieties, the binding partner(s) and/or the magnetic bead(s) by an electric (or electrical) field.

As used herein, “magnetic forces” are the forces exerted on moieties, the binding partner(s) and/or the magnetic bead(s) by a magnetic field.

As used herein, “sample” refers to anything which may contain a moiety to be transferred by the present microelectromagnetic dispenser heads and/or methods. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). Biological tissues may be processed to obtain cell suspension samples. The sample may also be a mixture of target analyte or enzyme containing molecules prepared in vitro. The sample may also be a cultured cell suspension. In case of the biological samples, the sample may be crude samples or processed samples that are obtained after various processing or preparation on the original samples. For example, various cell separation methods (e.g., magnetically activated cell sorting) may be applied to separate or enrich target cells from a body fluid sample such as blood. Samples used for the present invention include such target-cell enriched cell preparation.

As used herein, a “liquid (fluid) sample” refers to a sample that naturally exists as a liquid or fluid, e.g., a biological fluid. A “liquid sample” also refers to a sample that naturally exists in a non-liquid status, e.g., solid or gas, but is prepared as a liquid, fluid, solution or suspension containing the solid or gas sample material. For example, a liquid sample can encompass a liquid, fluid, solution or suspension containing a biological tissue.

As used herein the term “assessing (or assessed)” is intended to include quantitative and qualitative determination of the identity and/or quantity of a moiety, e.g., a protein or nucleic acid, present in the sample or on the magnetic beads or in whatever form or state. Assessment would involve obtaining an index, ratio, percentage, visual or other value indicative of the identity of a moiety in the sample and may further involve obtaining a number, an index, or other value indicative of the amount or quantity or the concentration of a moiety present in the sample or on the magnetic bead or in whatever form or state. Assessment may be direct or indirect. Assessment may be qualitative or quantitative.

B. Microelectromagnetic Dispenser Heads

In one aspect, the present invention is directed to a microelectromagnetic dispenser head, which head comprises: a) a core comprising a magnetizable substance, said core surrounded by a microcoil suitable for transmitting electrical current and generating a magnetic field via said magnetizable substance and said core having a tip suitable for attracting a magnetic or magnetically labeled moiety. Preferably, the microelectromagnetic dispenser head can further comprise one or both of the following: i) a shell that substantially shields magnetic field, generated via said microcoil, from the non-tip portion of said core; and/or ii) a cooling means for cooling said tip.

The core of the microelectromagnetic dispenser head can be in any suitable shape. For example, the core can be in the shape of a cylinder, cube or cuboid.

Any suitable magnetizable substance can be used as the core of the present microelectromagnetic dispenser heads. For example, paramagnetic substance, ferromagnetic substance and ferrimagentic substance can be used. In another example, the magnetizable substance used comprises a metal composition. In one specific embodiment, the metal composition used is a transition metal composition such as iron, nickel, copper, cobalt, manganese, tantalum, zirconium or an alloy thereof such as cobalt-tantalum-zirconium (CoTaZr) alloy. In another specific embodiment, the metal composition used is Fe₃O₄. Preferably, the magnetizable substance for the cores of the present microelectromagnetic dispenser heads is made of magnetically-soft materials. The magnetically-soft materials are the materials that can produce saturation of magnetization under a small applied field, and the magnetically-hard materials may require large applied field to produce saturation of magnetization. For example, soft magnetic alloys (e.g., 78 Permalloy, Ni: 78%, Fe: 22%) can be used as the material for the core. In another example, soft ferromagnetic wire (e.g., nickel) can be used as the material for the core.

Any suitable microcoil can be used in the present microelectromagnetic dispenser heads. For example, a microcoil can be made of 20 turns of copper wires having diameter of 25 microns. Such microcoils can readily conduct electric current of up to hundreds of mA. The core can be surrounded by a single microcoil. Alternatively, the core can be surrounded by a plurality of microcoils, e.g., three microcoils. The core can have a smooth surface and the microcoil can surround the core on the surface. Alternatively, the core can have certain structures, e.g., grooves, on its surface to accommodate the surrounding microcoils. In a specific embodiment, the core is movably surrounded by a microcoil and the relative positional movement between the core and the microcoil can be used to adjust the generated magnetic field. For example, the position of the core can be fixed in the microelectromagnetic dispenser heads and the microcoil is movable relative to the core. Alternatively, the position of the microcoil can be fixed in the microelectromagnetic dispenser heads and the core is movable relative to the microcoil.

The core and the tip can be assembled in any suitable fashion. For example, the head can comprise the core and the tip as an integral unit. Alternatively, the head can comprise the core and the tip as separate units. The core and the tip units can be assembled by any suitable connection, e.g., complementary grooves, gears, locks, etc. Preferably, the tip unit can be made as replaceable unit. When the microelectromagnetic dispenser heads are used to transfer different samples, such replaceable tips may be important in applications where cross contamination is prohibited or should be minimized. The core and the tip can comprise the same or different magnetizable substance(s).

The tip can be in any suitable shape. Preferably, the tip is in a sharp shape, e.g., needle, cylinder or circular cone, etc. The tip can have any suitable dimension comparable to the magnetic or magnetically labeled moiety to be attracted. For example, the tip can have a diameter ranging from about 100 to about 0.5 microns, e.g., about 100, from 100 to about 50, about, from about 50 to about 20, about 20, from about 20 to about 10, about 10, from about 10 to about 5, about 5, from about 5 to about 2, about 2, from 2 to about 1, about 1, from about 1 to about 0.5, about 0.5 or less than 0.5 micron(s). The surface of the tip can be in any suitable geometry, e.g., convex, concave or plane. Various methods can be used for preparing the shape of the tip, e.g., polishing, chemical or electrochemical etching. Depending on the magnetic or magnetically labeled moiety to be attracted and other operational considerations, the surface of the tip can be modified to be hydrophobic or hydrophilic. Other properties of the surface of the tip can be controlled and/or optimized. For example, the surface of the tip can be modified to comprise electrostatic charge. Any suitable surface modification or surface treatment methods can be used, for example, the surface of the tip can be coated with different polymer materials, or be treated in various chemical treatment solutions, or exposed to certain energy radiation such as laser, oxygen plasma.

The shell can be made of any suitable material so long that it can substantially shield magnetic field generated via said microcoil from the non-tip portion of the core. Preferably, the shell comprises a high permeability material that have high magnetic permeability and can efficiently conduct magnetic lines of force (or magnetic field). The ability to conduct magnetic lines of force (or magnetic field) is called permeability, and in a magnetic-shield material, the degree of permeability is expressed numerically. The standard or base line is free space with a rating of one, compared with high permeability material which ranges from about 5,000 to 350,000. Depending on the magnitude of the magnetic field to be shielded, high permeability materials with different permeability values and different magnetic saturation points may be used. For example, to shield a moderate magnetic field of 5,000 gauss, a high permeability material with permeability value of about 150,000 with a saturation point of about 7,500 gauss may be used. In another example, to shield a magnetic field of 12,000 gauss, a high permeability material with permeability value of about 80,000 with a saturation point of about 15,000 gauss may be used. Non-limiting examples of high permeability magnetic shielding material are a 80% nickel-iron-molybdenum alloy (nickel: 80%; molybdenum: 5%, iron and others: 15%), a 48% nickel-iron alloy (nickel: 48%, iron: 52%). Those who are skilled in magnetic shielding can readily choose appropriate high-permeability materials, depending on the magnetic fields generated by the microelectromagnetic dispenser head.

Any suitable cooling means can be used in the present microelectromagnetic dispensers or dispenser heads. The cooling means can be cooling materials and/or cooling devices. In one example, the cooling means used is a cooling material filled within the head. Any suitable cooling materials can be used, e.g., dry ice and liquid nitrogen. Such cooling materials filled within the head will effectively cool down the core of the dispenser head and cool down the tip. In addition, the cooling material can further comprise a high thermal conductivity materials that have high thermal conductivity and can efficiently conduct heat. Examples of high thermal conductivity materials include aluminum, copper, zinc, gold, silver, tungsten and silicon. Preferably, high thermal conductivity materials for cooling purposes have thermal conductivity over 1 J sec⁻¹ cm⁻¹° K.⁻¹ at room temperature (about 290-300° K.). In another example, the cooling means used is a cooling device, e.g., a Peltier cooler, associated with or integrated within the head. The cooling means can be placed in any suitable locations on the dispensers, e.g., filled within or associated with or integrated within the core and/or the tip portion of the head, or placed in the non-head portion of the dispensers. Via thermal conduction, these cooling means will result in a cooling effect on the tips. In one specific embodiment, the cooling means is attached to the head and is in contact with both ends of the microcoil. In another specific embodiment, high thermal conductivity materials are used as the cooling material and are in contact with the microcoil(s) of the dispenser head on one hand and in contact with a low temperature source on the other hand. In this way, the heat generated in the microcoil(s) due to the application of DC current can be rapidly dissipated via the high-thermal conductivity material path to the low temperature source.

The present microelectromagnetic dispenser head can comprise any suitable number of the tip(s). For example, the head can comprise a single tip. Alternatively, the head can comprise a plurality of the tips. When the head comprises a plurality of the tips, the distance among the tips preferably corresponds to the distance among the magnetic or magnetically labeled moieties to be attracted, e.g., magnetically labeled moieties located in multiples wells of a microtiter plate.

In a preferred embodiment, the present microelectromagnetic dispenser head comprises both a shell that substantially shields magnetic field, generated via the microcoil, from the non-tip portion of the core, and a cooling means for cooling the tip.

An array of microelectromagnetic dispenser heads, which array comprises a plurality of the above microelectromagnetic dispenser heads, are also provided. Some or all of the microelectromagnetic dispenser heads within the array can be made individually addressable. Preferably, each of the microelectromagnetic dispenser head is individually addressable. The microelectromagnetic dispenser heads can be made individually addressable by any suitable means. For example, U.S. patent application Ser. No. 09/685,410, filed on Oct. 10, 2000, entitled “Individually Addressable Micro-Electrormagnetic Unit Array Chips in Horizontal Configurations” discloses various means for individually addressing electromagnetic units on a chip and these means can also be used for individually addressing the microelectromagnetic dispenser heads.

A microelectromagnetic dispenser, which dispenser comprises: a) a present microelectromagnetic dispenser head; and b) a means for controllably moving said head, is also provided. The present microelectromagnetic dispenser head in the dispenser can comprise a single or a plurality of the tips. In addition, a microelectromagnetic dispenser, which dispenser comprises: a) an array of the present microelectromagnetic dispenser heads; and b) a means for controllably moving said array, is further provided. Some or all of the microelectromagnetic dispenser heads within the array can be made individually addressable. Preferably, each of the microelectromagnetic dispenser head is individually addressable

C. Methods for Transferring Moieties

In another aspect, the present invention is directed to a method for transferring a moiety, which method comprises: a) providing a magnetic or magnetically labeled moiety to be transferred at first location, b) providing a microelectromagnetic dispenser head comprising a core comprising a magnetizable substance, said core surrounded by a microcoil suitable for transmitting electrical current and generating a magnetic field via said magnetizable substance and said core having a tip suitable for attracting said magnetic or magnetically labeled moiety; c) attracting said magnetic or magnetically labeled moiety from said first location to said tip of said microelectromagnetic dispenser head; and d) transferring and releasing said magnetic or magnetically labeled moiety to a second location from said tip of said microelectromagnetic dispenser head.

Any moiety can be transferred by the present methods. Intrinsically magnetic moiety can transferred by the present method directly. For example, magnetic moiety may be a magnetic bead to which bioanalytes such as bio-molecules are attached. Intrinsically non-magnetic moiety can be made magnetic, e.g., by attaching to a magnetic particle (e.g. a magnetic bead of 5 micron in diameter), and transferred by the present method. Exemplary moieties that can be transferred by the present method include cells, cellular organelles, viruses, molecules and an aggregate or complex thereof.

Moieties to be transferred can be pure substances or can exist in a mixture of substances wherein the target moiety is only one of the substances in the mixture. For example, cancer cells in the blood from leukemia patients, cancer cells in the solid tissues from patients with solid tumors and fetal cells in maternal blood from pregnant women can be selectively made magnetic by, for example, binding target cells to magnetic beads whose surfaces have been immobilized with antibodies against the target cells and be transferred. In these examples, the blood from leukemia patients, solid tissues from patients with solid tumors and maternal blood from pregnant women may have to be processed to enrich the target cells by specific cell enrichment or cell separation procedures. Similarly, various blood cells such as red and white blood cells in the blood can be selectively made magnetic and be transferred. DNA molecules, mRNA molecules, certain types of protein molecules, or all protein molecules from a cell lysate can be moieties to be transferred.

A moiety can be attached to a magnetic particle by specific binding or non-specific binding. Preferably, a moiety is attached to a magnetic particle via a binding partner on the magnetic particle that is capable of specifically binding to a moiety to be transferred. Depending on the moiety to be attached and transferred, a binding partner can be any suitable substance, e.g., a cell, cellular organelle, virus, molecule and an aggregate or complex thereof. Preferably, the binding partner is an antibody or a nucleotide sequence. A magnetic particle can comprise a single binding partner. Alternatively, a magnetic particle can comprise a plurality of binding partners, each binding partner is capable of binding or specifically binding to a different moiety.

Cells can be moieties to be transferred or to be used as binding partners. Non-limiting examples of cells include animal cells, plant cells, fungi, bacteria, recombinant cells or cultured cells. Animal, plant cells, fungus, bacterium cells to be transferred or to be used as binding partners can be derived from any genus or subgenus of the Animalia, Plantae, fungus or bacterium kingdom. Cells derived from any genus or subgenus of ciliates, cellular slime molds, flagellates and microsporidia can also be transferred or be used as binding partners. Cells derived from birds such as chickens, vertebrates such as fish and mammals such as mice, rats, rabbits, cats, dogs, pigs, cows, ox, sheep, goats, horses, monkeys and other non-human primates, and humans can also be transferred or be used as binding partners.

For animal cells, cells derived from a particular tissue or organ can be transferred or be used as binding partners. For example, connective, epithelium, muscle or nerve tissue cells can be transferred or be used as binding partners. Similarly, cells derived from an accessory organ of the eye, annulospiral organ, auditory organ, Chievitz organ, circumventricular organ, Corti organ, critical organ, enamel organ, end organ, external female genital organ, external male genital organ, floating organ, flower-spray organ of Ruffini, genital organ, Golgi tendon organ, gustatory organ, organ of hearing, internal female genital organ, internal male genital organ, intromittent organ, Jacobson organ, neurohemal organ, neurotendinous organ, olfactory organ, otolithic organ, ptotic organ, organ of Rosenmüller, sense organ, organ of smell, spiral organ, subcommissural organ, subfornical organ, supernumerary organ, tactile organ, target organ, organ of taste, organ of touch, urinary organ, vascular organ of lamina terminalis, vestibular organ, vestibulocochlear organ, vestigial organ, organ of vision, visual organ, vomeronasal organ, wandering organ, Weber organ and organ of Zuckerkandl can be transferred or be used as binding partners. Preferably, cells derived from an internal animal organ such as brain, lung, liver, spleen, bone marrow, thymus, heart, lymph, blood, bone, cartilage, pancreas, kidney, gall bladder, stomach, intestine, testis, ovary, uterus, rectum, nervous system, gland, internal blood vessels, etc., can be transferred or be used as binding partners. Further, cells derived from any plants, fungi such as yeasts, bacteria such as eubacteria or archaebacteria can be transferred or be used as binding partners. Recombinant cells derived from any eucaryotic or prokaryotic sources such as animal, plant, fungus or bacterium cells can also be transferred or be used as binding partners. Cells from various types of body fluid such as blood, urine, saliva, bone marrow, sperm or other ascitic fluids, and subfractions thereof, e.g., serum or plasma, can also be transferred or be used as binding partners.

Cellular organelles including nucleus, mitochondria, chloroplasts, ribosomes, ERs, Golgi apparatuses, lysosomes, proteasomes, secretory vesicles, vacuoles or microsomes, can be transferred or be used as binding partners. Viruses including intact viruses or any viral structures, e.g., viral particles, in the virus life cycle that can be derived from viruses such as Class I viruses, Class II viruses, Class III viruses, Class IV viruses, Class V viruses or Class VI viruses, can also be transferred or be used as binding partners.

Inorganic molecules such as ions, and organic molecules or a complex thereof, can be transferred or be used as binding partners. Non-limiting examples of ions include sodium, potassium, magnesium, calcium, chlorine, iron, copper, zinc, manganese, cobalt, iodine, molybdenum, vanadium, nickel, chromium, fluorine, silicon, tin, boron or arsenic ions. Non-limiting examples of organic molecules include amino acids, peptides, proteins, nucleosides, nucleotides, oligonucleotides, nucleic acids, vitamins, monosaccharides, oligosaccharides, carbohydrates, lipids or a complex thereof.

Any amino acids can be transferred or be used as binding partners. For example, a D- and a L-amino-acid can be transferred or be used as binding partners. In addition, any building blocks of naturally occurring peptides and proteins including Ala (A), Arg (R), Asn (N), Asp (D), Cys (C), Gln (Q), Glu (E), Gly (G), His (H), Ile (I), Leu (L), Lys (K), Met (M), Phe (F), Pro (P) Ser (S), Thr (T), Trp (W), Tyr (Y) and Val (V) can be transferred or be used as binding partners.

Any proteins or peptides can be transferred or be used as binding partners. For example, membrane proteins such as receptor proteins on cell membranes, enzymes, transport proteins such as ion channels and pumps, nutrient or storage proteins, contractile or motile proteins such as actins and myosins, structural proteins, defense protein or regulatory proteins such as antibodies, hormones and growth factors can be transferred or be used as binding partners. Proteineous or peptidic antigens can also be transferred or be used as binding partners.

Any nucleic acids, including single-, double and triple-stranded nucleic acids, can be transferred or be used as binding partners. Examples of such nucleic acids include DNA, such as A-, B- or Z-forn DNA, and RNA such as mRNA, tRNA and rRNA.

Any nucleosides can be transferred or be used as binding partners. Examples of such nucleosides include adenosine, guanosine, cytidine, thymidine and uridine. Any nucleotides can be isolated, manipulated or detected by the present methods. Examples of such nucleotides include AMP, GMP, CMP, UMP, ADP, GDP, CDP, UDP, ATP, GTP, CTP, UTP, dAMP, dGMP, dCMP, dTMP, dADP, dGDP, dCDP, dTDP, dATP, dGTP, dCTP and dTTP.

Any vitamins can be transferred or be used as binding partners. For example, water-soluble vitamins such as thiamine, riboflavin, nicotinic acid, pantothenic acid, pyridoxine, biotin, folate, vitamin B₁₂ and ascorbic acid can be transferred or be used as binding partners. Similarly, fat-soluble vitamins such as vitamin A, vitamin D, vitamin E, and vitamin K can be transferred or be used as binding partners.

Any monosaccharides, whether D- or L-monosaccharides and whether aldoses or ketoses, can be transferred or be used as binding partners. Examples of monosaccharides include triose such as glyceraldehyde, tetroses such as erythrose and threose, pentoses such as ribose, arabinose, xylose, lyxose and ribulose, hexoses such as allose, altrose, glucose, mannose, gulose, idose, galactose, talose and fructose and heptose such as sedoheptulose.

Any lipids can be transferred or be used as binding partners. Examples of lipids include triacylglycerols such as tristearin, tripalmitin and triolein, waxes, phosphoglycerides such as phosphatidylethanolamine, phosphatidylcholine, phosphatidylserine, phosphatidylinositol and cardiolipin, sphingolipids such as sphingomyelin, cerebrosides and gangliosides, sterols such as cholesterol and stigmasterol and sterol fatty acid esters. The fatty acids can be saturated fatty acids such as lauric acid, myristic acid, palmitic acid, stearic acid, arachidic acid and lignoceric acid, or can be unsaturated fatty acids such as palmitoleic acid, oleic acid, linoleic acid, linolenic acid and arachidonic acid.

In a specific embodiment, the microelectromagnetic dispenser head can further comprise a shell that substantially shields magnetic field, generated via the microcoil, from the non-tip portion of the core of the microelectromagnetic dispenser head. The shell can be made of any suitable material so long that it can substantially shield magnetic field generated via said microcoil from the non-tip portion of the core. Preferably, the shell comprises a high permeability material that have high magnetic permeability and can efficiently conduct magnetic lines of force (or magnetic field). Non-limiting examples of high permeability magnetic shielding material are a 80% nickel-iron-molybdenum alloy (nickel: 80%; molybdenum: 5%, iron and others: 15%), a 48% nickel-iron alloy (nickel: 48%, iron: 52%). Those who are skilled in magnetic shielding can readily choose appropriate high-permeability materials, depending on the magnetic fields generated by the microelectromagnetic dispenser head.

In another specific embodiment, the microelectromagnetic dispenser head is part of a microelectromagnetic dispenser that further comprises a means for controllably moving the head. In one example, the dispenser head is attached to a 3-dimensional motion stage (e.g., motorized precision positioner or 3-D micromanipulator) so that the head can be moved by controlling and moving the motion stage. Such motion stage can have accurate, sub-micron resolution and can be controlled electronically or manually.

The present methods transfer moieties via magnetic force, alone or in combination with other types of forces. Magnetic forces refer to the forces acting on a moiety or particle due to the application of a magnetic field. In general, moieties have to be magnetic or magnetically labeled to be transferred. When the magnetic or magnetically labeled moiety is subjected to a magnetic field \overline{B}, a magnetic dipole \overline{μ} is induced in the magnetic or magnetically labeled moiety

$$\begin{matrix}\overline{μ} = {V}_{p} ( {χ}_{p} - {χ}_{m} ) \frac{\overline{B}}{{μ}_{m}} , \\ = {V}_{p} ( {χ}_{p} - {χ}_{m} ) {\overline{H}}_{m}\end{matrix}$$

where Vₚ is the volume of the magnetic or magnetically labeled moiety, χₚ and χₘ are the volume susceptibility of the magnetic or magnetically labeled moiety and its surrounding medium, μₘ is the magnetic permeability of medium, \overline{H}ₘ is the magnetic field strength. The magnetic force \overline{F}_{magnetic} acting on the magnetic or magnetically labeled moiety is determined by the magnetic dipole moment and the magnetic field gradient:

\overline{F}_{magnetic}=−0.5Vₚ(χₚ−χₘ)\overline{H}ₘ·∇\vec{B}ₘ,

where the symbols “●” and “∇” refer to dot-product and gradient operations, respectively. Clearly, whether there is magnetic force acting on a moiety depends on the difference in the volume susceptibility between the magnetic or magnetically labeled moiety and its surrounding medium. Typically, magnetic or magnetically labeled moieties are suspended in a liquid, non-magnetic medium (the volume susceptibility is close to zero) thus it is necessary to utilize magnetic or magnetically labeled moieties (its volume susceptibility is much larger than zero). The velocity ν_{particle} of the magnetic or magnetically labeled moiety under the balance between magnetic force and viscous drag is given by:

$${v}_{particle} = \frac{{\overline{F}}_{magnetic}}{6 π r {η}_{m}}$$

where r is the moiety or particle radius and ηₘ is the viscosity of the surrounding medium. Thus to achieve sufficiently large magnetic force, the following factors should be considered: (1) the volume susceptibility of the magnetic or magnetically labeled moietiess should be maximized; (2) magnetic field strength should be maximized; and (3) magnetic field strength gradient should be maximized.

The magnetic field in the attracting, transferring and/or releasing step(s) can be adjusted, e.g., augmented, countered or removed. Such adjustment can be effected in any suitable ways. In one example, the magnetic field is adjusted by a relative positional movement between the core and the microcoil of the microelectromagnetic dispenser head. The position of the core can be fixed in the microelectromagnetic dispenser heads and the microcoil is movable relative to the core. Alternatively, the position of the microcoil can be fixed in the microelectromagnetic dispenser heads and the core is movable relative to the microcoil. The magnetic field can also be adjusted by adjusting electric current in the microcoil, e.g., by changing the polarity and/or magnitude of the electric current, or by shutting off the electric current in the microcoil. The magnetic field can further be adjusted by an external augmentative or counter magnetic field, e.g., the external augmentative or counter magnetic field effected via an external magnet or electromagnetic unit. In a specific embodiment, the magnetic or magnetically labeled moiety is released by shutting off the electric current in the microcoil. In another embodiment, the magnetic or magnetically labeled moiety is released by turning on an external electromagnetic unit to produce a magnetic field that is in the opposite direction to the magnetic field generated by the dispenser head. Still, in another embodiment, the magnetic or magnetically labeled moiety is released by moving an external magnet to a nearby location of the dispenser head so that the external magnet produces a magnetic field that is in the opposite direction to the magnetic field generated by the dispenser head and is sufficiently strong to separate the magnetic or magnetically labeled moiety from the dispenser head tip.

The magnetic or magnetically labeled moiety in the attracting, transferring and/or releasing step(s) can be cooled. The cooling can be achieved by any suitable means. The cooling means can be cooling materials and/or cooling devices. In one example, the cooling means used is a cooling material filled within the dispenser, e.g., in the head portion. Any suitable cooling materials can be used, e.g., dry ice and liquid nitrogen. In addition, the cooling material can further comprise a high thermal conductivity material that has high thermal conductivity and can efficiently conduct heat. Examples of high thermal conductivity materials include aluminum, copper, zinc, gold, silver, tungsten and silicon. Preferably, high thermal conductivity materials for cooling purposes have thermal conductivity over 1 J sec⁻¹ cm⁻¹° K.⁻¹ at room temperature (about 290-300° K.). In another example, the cooling means used is a cooling device,.e.g., a Peltier cooler, associated with or integrated within the dispenser, e.g., in the head portion. The cooling means can be placed in any suitable place, e.g., filled within or associated with or integrated within the core and/or the tip portion of the head, or other suitable non-head portion of the dispenser. In one specific embodiment, the cooling means is attached to the head and is in contact with both ends of the microcoil.

In a specific embodiment, the magnetic or magnetically labeled moiety at the first location is in a liquid state in a liquid container and is cooled to be in a solid state in the attracting, transferring and/or releasing step(s). The present method can further comprise a heating step to reduce the adhesion of the magnetic or magnetically labeled moiety (in its solid state) to the container and to facilitate attracting, transferring and/or releasing of the magnetic or magnetically labeled moiety, said heating step does not change the magnetic or magnetically labeled moiety from solid state to liquid state. The heating can be effected via any suitable means, e.g., an external heater or an internal heating unit associated or integrated with the dispenser or dispenser head.

The present method can be used to transfer some or all magnetic or magnetically labeled moieties from a first location to a second location. In a specific embodiment, the present method is used to transfer all magnetic or magnetically labeled moieties from a first location to a second location.

The present method can further comprise identifying the magnetic or magnetically labeled moieties containing a non-magnetic, identifiable signal and attracting, transferring and/or releasing such identified magnetic or magnetically labeled moieties from a first location to a second location. Preferably, the non-magnetic, identifiable signal is an optical signal effected by an optical labeling substance comprised in a magnetic particle. For example, the moiety is a biological cell that has been magnetically labeled by binding the cell with a magnetic bead. The target cells to be transferred are the cells that are labeled with fluorescent molecules that are indicative of specific property of the target cells. In this case, the present method further comprise a step for identifying the cells that produce fluorescent signals, followed by attracting, transferring and/or releasing such identified cells from a first location to a second location.

Any suitable optical labeling substance can be used in the present methods or magnetic particles. In specific embodiments, the optical labeling substance used in the present methods or magnetic particles is a fluorescent substance, a scattered-light detectable particle (See e.g., U.S. Pat. No. 6,214,560) and a quantum dot (See e.g., U.S. Pat. No. 6,252,664).

Any suitable quantum dot can be used in the present methods or magnetic particles. In a specific embodiment, the quantum dot used in the present methods or magnetic particles comprises a Cd-X core, X being Se, S or Te. Preferably, the quantum dot can be passivated with an inorganic coating shell, e.g., a coating shell comprising Y-Z, Y being Cd or Zn, and Z being S or Se. Also preferably, the quantum dot can comprise a Cd-X core, X being Se, S or Te, a Y-Z shell, Y being Cd or Zn, and Z being S or Se, and the particle can further be overcoated with a trialkylphosphine oxide.

Any suitable methods can be used to make the CdX core/YZ shell quantum dots water-soluble (See e.g., U.S. Pat. No. 6,252,664). One method to make the CdX core/YZ shell quantum dots water-soluble is to exchange this overcoating layer with a coating which will make the quantum dots water-soluble. For example, a mercaptocarboxylic acid may be used to exchange with the trialkylphosphine oxide coat. Exchange of the coating group is accomplished by treating the water-insoluble quantum dots with a large excess of neat mercaptocarboxylic acid. Alternatively, exchange of the coating group is accomplished by treating the water-insoluble quantum dots with a large excess of mercaptocarboxylic acid in CHCl₃ solution (Chan and Nie, Science, 281:2016-2018 (1998)). The thiol group of the new coating molecule forms Cd (or Zn)-S bonds creates a coating which is not easily displaced in solution. Another method to make the CdX core/YZ shell quantum dots water-soluble is by the formation of a coating of silica around the dots (Bruchez et al., Science, 281:2013-2015 (1998)). An extensively polymerized polysilane shell imparts water solubility to nanocrystalline materials, as well as allowing further chemical modifications of the silica surface. Generally, these “water-soluble” quantum dots require further functionalization to make them sufficiently stable in an aqueous solution for practical use in a fluorescence detection system (See e.g., U.S. Pat. No. 6,114,038), particularly when exposed to air (oxygen) and/or light. Water-soluble functionalized nanocrystals are extremely sensitive in terms of detection, because of their fluorescent properties (e.g., including, but not limited to, high quantum efficiency, resistance to photobleaching, and stability in complex aqueous environments); and comprise a class of semiconductor nanocrystals that may be excited with a single peak wavelength of light resulting in detectable fluorescence emissions of high quantum yield and with discrete fluorescence peaks (e.g., having a narrow spectral band ranging between about 10 nm to about 60 nm).

The quantum dot used in the present methods or magnetic particles can have any suitable size. For example, the quantum dot can have a size ranging from about 1 nm to about 100 nm.

The magnetic particles used in the present methods can comprise a single quantum dot. Alternatively, the magnetic particles used in the present methods can comprise a plurality of quantum dots. Preferably, the magnetic particles used in the present methods comprises at least two quantum dots that have different sizes and/or different colors. Details of quantum dots and how they can be incorporated into the magnetic particles may be found in the literatures, for example, in the articles by Chan and Nie, Science, 281:2016 (1998) and by Han et al., Nature Biotehnology, 19:631-635 (2001).

The magnetic particles used in the present methods can comprise a single optical labeling substance. Alternatively, the magnetic particles used in the present methods can comprise a plurality of optical labeling substances.

The present method can be used to transfer a moiety between or among any desired locations. Exemplary locations include a beaker, a flask, a cylinder, a test tube, an enpindorf tube, a centrifugation tube, a culture dish, a multiwell plate, a filter membrane, a microscopic slide and a chip. Any suitable chips, for example, DNA microarray membrane and DNA microarray glass or silicon chip, can be used in the present method. For example, the active chips comprising multiple force generating elements disclosed in the U.S. patent application Ser. No. 09/679,024 can be used in the present methods.

The present method can be used in high throughput mode by using a microelectromagnetic dispenser head comprising a plurality of the tips to attract, transfer and/or release a plurality of the magnetic or magnetically labeled moieties from a first plurality of locations to a second plurality of locations. Similarly, the present method can be used in high throughput mode by using an array of the microelectromagnetic dispenser heads to attract, transfer and/or release a plurality of the magnetic or magnetically labeled moieties from a first plurality of locations to a second plurality of locations.

In a specific embodiment, a magnetically labeled moiety is transferred from a first location to a second location and the present method further comprises recovering said transferred moiety from said magnetic label, e.g., by optical, chemical or other cleavage methods.

D. Preferred Embodiment

In one embodiment, the present invention is directed to a microelectromagnetic dispenser, which microelectromagnetic dispenser can transport and dispense magnetic moieties or particles, or magnetically labeled moieties or particles efficiently and quickly. The microelectromagnetic dispenser can be used for solid particles or liquid sample dispensation and the cleaning procedures are very simple.

In another embodiment, the present invention is directed to a microelectromagnetic dispenser array, which microelectromagnetic dispenser array can transport and dispense many different kinds of microparticles at the same time. Also this kind of microelectromagnetic dispenser array can transport and dispense magnetic moieties or particles efficiently and quickly and can be used for solid particles or liquid sample dispensation. The use of the present microelectromagnetic dispenser array makes the transfer efficiency higher and the cleaning procedures simpler.

In another embodiment, the present invention is directed to a method for quickly and efficiently transporting and dispensing the magnetic moieties or particles, or magnetically labeled moieties or particles. The method can be used for transporting and dispensing solid particles or liquid sample with high efficiency.

In another embodiment, the present invention is directed to a method for quickly and efficiently transporting and dispensing a liquid sample, which method has high efficiency.

One exemplary microelectromagnetic dispenser can comprise: a) a magnetic head; b) a signal source; and c) a shell shielding magnetic force from the non-tip portion of the head. The magnetic head includes a magnetic core with a sharp tip for attracting the magnetic particles, and a microcoil that comprises a wire that winds over the magnetic core. The signal source is used to provide the electrical current to the wire of the microcoil. And the shell that covers the wire and magnetic core is used to shield the magnetic field.

The microelectromagnetic dispenser can also have a cooling system, which can decrease the temperature at the tip of the dispenser. This kind of microelectromagnetic dispenser can be used to manipulate the low temperature solid particle and keep the particle at low temperature. The cooling system in the microelectromagnetic dispenser can be an external cooling device, integrated in the microelectromagnetic dispenser, or a hole on the magnetic core and filled with a cooling material, or high thermal conductivity materials in contact with the dispenser head and in contact with a low temperature source.

One exemplary microelectromagnetic dispenser array comprises several said microelectromagnetic dispensers. Each microelectromagnetic dispenser has a magnetic head, a signal source and a shell shielding magnetic field or magnetic line of force from the non-tip portion of the head. And these microelectromagnetic dispensers are individual addressable. Another exemplary microelectromagnetic dispenser array comprises a big magnetic head with a tip array.

One exemplary moiety transferring method using the microelectromagnetic dispenser includes: a) using microelectromagnetic dispenser to generate a magnetic field or force and attract the magnetic particle to the dispenser head, moving the particle to target location; b) withdrawing the magnetic force and dropping the magnetic particle. In this method, since the current in the microcoil can be controlled, the magnitude of magnetic field or force can be adjusted to attract the specific magnetic particles. Based on the number of the magnetic particles to be attracted and the weight of each magnetic particle, different magnetic force can be used. Another way to adjust the magnitude of the magnetic force without controlling the current is: a) setting the current to a specific value; b) fixing the microcoil, manipulating the magnetic head up and down to generate the appropriate magnetic force; or c) fixing the magnetic head, manipulating the microcoil up and down to generate the appropriate magnetic force.

To facilitate the magnetic particles to be dropped on the target location, an external magnet under the target location can be used to attract the magnetic particle after withdrawing the magnetic force on microelectromagnetic dispenser. An alternative approach is to use an electromagnetic element that can be placed under the target location. After turning off the microelectromagnetic dispenser and withdrawing the magnetic force, the electromagnetic element can be turned on to attract the magnetic particle.

Another exemplary liquid moiety transferring method using the microelectromagnetic dispenser includes: a) using microelectromagnetic dispenser to generate a magnetic field or force and attract the magnetic bead to the dispenser head, placing the magnetic bead to sample solution; b) cooling the sample solution so that the sample and magnetic bead become a solidified, mixed, cool magnetic particle; c) using microelectromagnetic dispenser to generate the magnetic force and attract the solid, mixed, cool magnetic particle, transporting this magnetic particle to target location; d) withdrawing the magnetic force and dropping the magnetic particle. In this method, the sample solution mixes with magnetic bead and becomes a solidified, magnetic particle, which can be manipulated and dispensed by microelectromagnetic dispenser.

In one specific embodiment of the present invention, the microelectromagnetic dispenser comprises: a) a magnetic core fabricated using a soft magnetic material; b) a microcoil comprising electric wires wound over the magnetic core; c) a signal source to provide an electrical current to the microcoil, in which the current can be controlled to generate magnetic field or force of different magnitude; and d) a shell covering the whole magnetic head, including the magnetic core and microcoil, which shell can shield the magnetic field from the non-tip portion of the head. The magnetic core has a needle type tip and a cylindrical body, which is wound with the microcoil. A hole can be drilled in the cylindrical body and filled with cooling material, such as liquid nitrogen and dry ice, to keep the whole microelectromagnetic dispenser or the head portion at low temperature. Alternatively, a Peltier cooler can be used to keep the whole microelectromagnetic dispenser or the head portion at low temperature. With these cooling systems, the microelectromagnetic dispenser can handle low temperature sample.

There are two steps to dispense a liquid sample using the microelectromagnetic dispenser. First, the microelectromagnetic dispenser is used to dispense magnetic beads to sample chambers on the sample container (such as 96 wells plate). The magnetic force generated by the microelectromagnetic dispenser can be controlled to handle the magnetic beads. Each chamber should have one magnetic bead dispensed therein. After cooling the sample chambers, the sample solution and the magnetic bead become a solidified, mixed, cool magnetic particle. Second, the mixed, cool magnetic particle is dispensed to a target location. The mixed, cool magnetic particle can be attracted and transported by the microelectromagnetic dispenser. After the magnetic particles are transported to the target location, the magnetic field or force from the microelectromagnetic dispenser is withdrawn and the magnetic particles are dropped to the target locations. If there is remanence on the microelectromagnetic dispenser and the magnetic particles cannot be dropped, an external magnet can be placed under the sample container or chip to generate a strong magnetic force and attract the magnetic particles. In this way, the sample handled by microelectromagnetic dispenser is a solidified sample and will not stay on the microelectromagnetic dispenser. Thus, it is easy to clean the microelectromagnetic dispenser and to avoid or minimize contamination. Combined with precision robot, the microelectromagnetic dispenser can quickly and efficiently transport and dispense many kinds of samples.

The tips of the microelectromagnetic dispenser can have different shapes, such as needle, cylinder and so on, which suit to attract and transport microparticles. Also, the surface of the tip can be convex, concave, or plane.

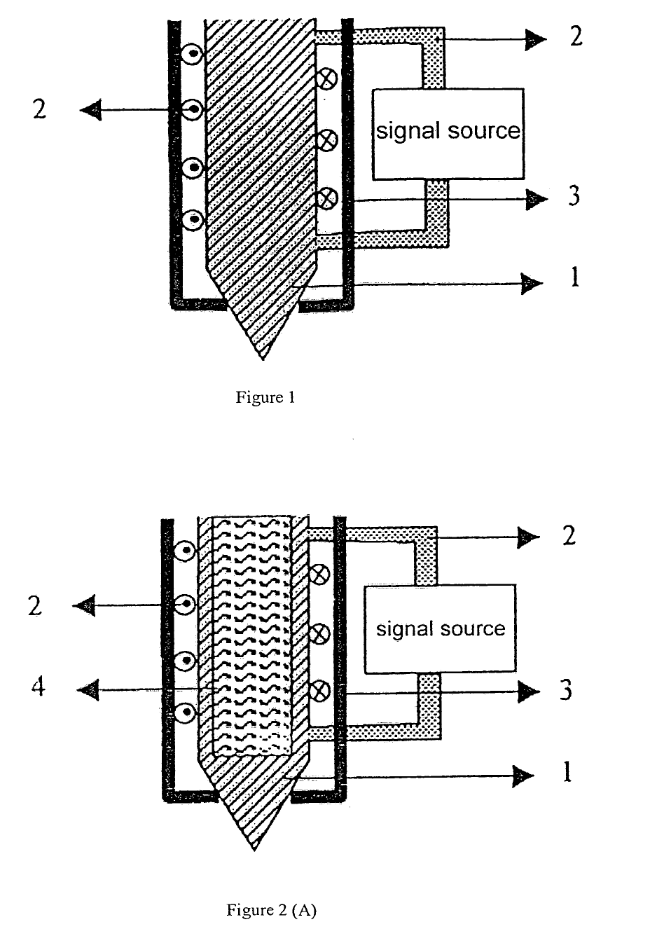

FIG. 1 shows one example of the present microelectromagnetic dispenser head. The microelectromagnetic dispenser head comprises a magnetic core 1, a microcoil 2 comprising an electric wire that winds over the magnetic core and a shell 3, which can shield magnetic field from the non-tip portion of the head. The magnetic core 1 has a needle type tip. The material of the magnetic core 1 is soft magnetic material. Only when a current is applied to the microcoil 2, the magnetic core 1 will induce a magnetic field. If there is no current in the microcoil 2, the magnetic core 1 will not induce a magnetic field. The magnetic core may have very small magnetic remanence. The magnetic core 1 is wound with the microcoil 2, which is used to induce the magnetic field. The shell 3 that covers the microcoil 2 and magnetic core 1 is used to shield magnetic field from the non-tip portion of the head. The material of shell 3 is special, high permeability material that can shield a magnetic field. The current that is applied to the microcoil 2 will control the generation of the magnetic field or force. The magnetic field around the tip can be adjusted through controlling the magnitude of the current in the microcoil 2. Also this magnetic field can be adjusted by other methods, such as by fixing the microcoil 2, manipulating the magnetic core 1 up and down; or fixing the magnetic core 1, and manipulating the microcoil 2 up and down.

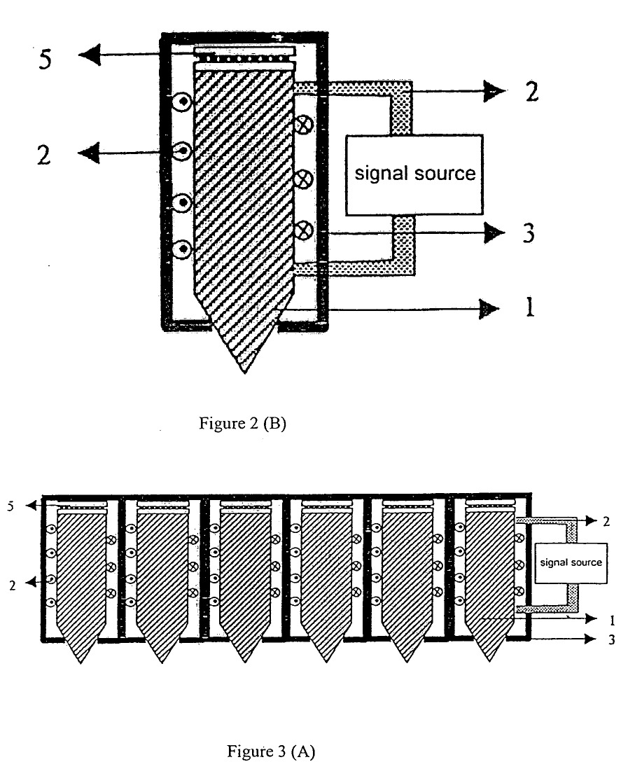

FIG. 2 shows two examples of the present microelectromagnetic dispenser head with a cooling system. One purpose of the present microelectromagnetic dispenser head is to transport and dispense low temperature solid sample. So not only the whole system need to be kept at low temperature environment, but also the head of microelectromagnetic dispenser need to have a low temperature. In one embodiment of the present invention, the microelectromagnetic dispenser has a cooling system. In FIG. 2 (A), the magnetic core has a hole, which is filled with a cooling material, such as dry ice and liquid nitrogen, to keep the microelectromagnetic dispenser at low temperature. In FIG. 2 (B), an external cooling device 5 (e.g., Peltier cooler) is placed on top of magnetic core 1. After applying an electrical signal to this cooling device 5, a low temperature can be generated on the cooling device surface, which is contact with the magnetic core to keep the magnetic core at low temperature.

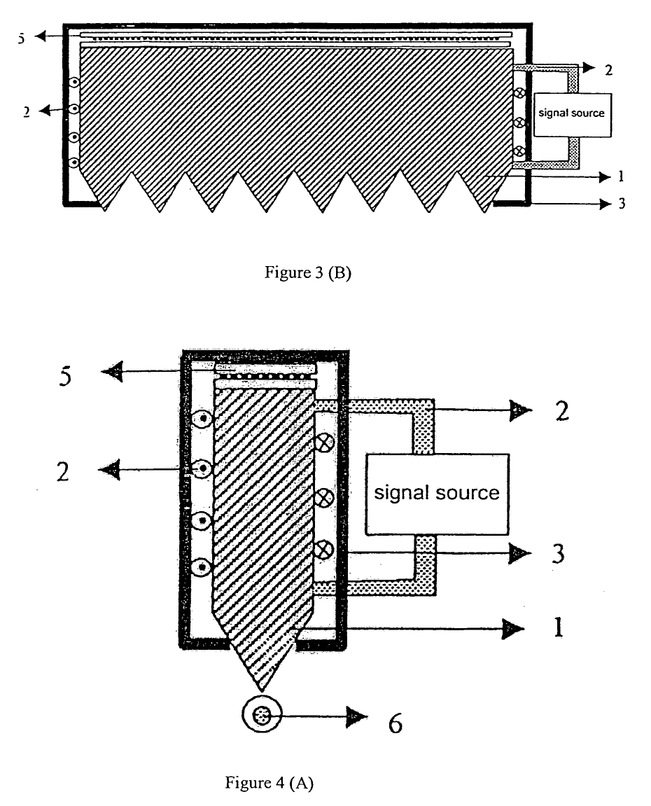

FIG. 3 shows two examples of the present microelectromagnetic dispenser head array. This microelectromagnetic dispenser head array can transport and dispense many different kinds of microparticles at the same time and increase the efficiency. In FIG. 3 (A), the microelectromagnetic dispenser head array comprises several said microelectromagnetic dispensers. Each microelectromagnetic dispenser head has magnetic core 1, microcoil 2, shell 3, and cooling device 5. Each microelectromagnetic dispenser head is individual addressable. The distance between each microelectromagnetic dispenser head equals to the distance between each well on the sample container (e.g., 96 wells plate). Thus, it is easy to simultaneously transport multiple samples. Also the microelectromagnetic dispenser head is individual addressable, so the samples on the different microelectromagnetic dispenser heads can be dispensed at different times and different locations. The microelectromagnetic dispenser head array in FIG. 3 (A) has 6 microelectromagnetic dispenser heads. In FIG. 3 (B), the microelectromagnetic dispenser head array has one set of microcoil 2, shell 3 , and a big magnetic core, which has a tip array. Using this tip array, many samples can be transported, but these samples can be dispensed only simultaneously. The magnetic core in FIG. 3 (B) has 8 tips.

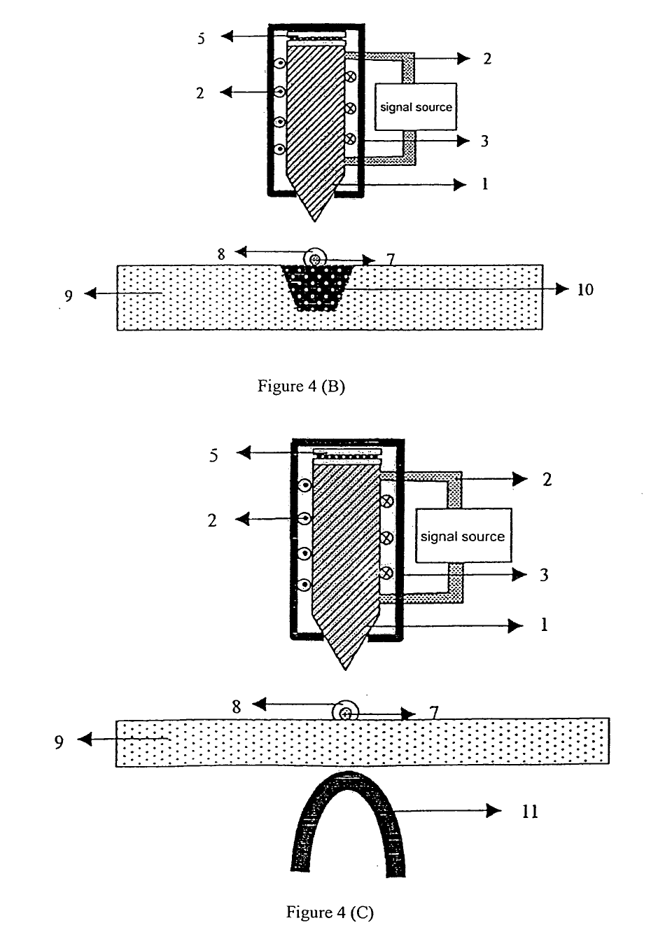

FIG. 4 shows the schematic procedures of a liquid sample handling method with the present microelectromagnetic dispenser head. There are two steps to dispense the liquid sample using the present microelectromagnetic dispenser head. First, using microelectromagnetic dispenser head to dispense the magnetic beads to each sample. The diameter of the magnetic beads is between 1 μm to 1 cm. The magnetic force generated by the microelectromagnetic dispenser head can be controlled to handle the magnetic beads. The number of magnetic beads transported by the microelectromagnetic dispenser head and the number of the magnetic beads in each kind of the sample solution can be controlled. The sample solutions are in the different wells of the sample container (e.g., 96-well plate, 384-well plate, or 1536 well plate). The surfaces of these wells are treated to be hydrophobic, so the shape of the sample solution is like a spherical ball or near-sphere ball. After cooling the sample container, the sample solution becomes a solidified sample particle. The contact surface area of the solidified sample particle and sample container is small. This small area will result in small resistance or small adhesion/binding force between the solidified sample particle and sample container when microelectromagnetic dispenser attracts the solid sample particle. If the solid sample particle sticks to the sample container, an external heater can be used to melt the surface of solid sample particle, which will decrease the adhesion/binding force between magnetic particle and sample container. After the sample solution in the sample container mixes with magnetic beads and cooling the sample container, the sample solution and magnetic beads will become a solidified, sample-magnetic bead complex 6. Since this complex 6 has magnetic beads inside, it is easy to be transported and dispensed by the present microelectromagnetic dispenser head. FIG. 4 (A) shows the microelectromagnetic dispenser head attracting a solidified, sample-magnetic bead complex 6. After using precision robot to move the microelectromagnetic dispenser head to a target location, the current in the microcoil 2 can be turned off to withdraw the magnetic field or force and to drop the solidified, sample-magnetic bead complex 6 to the target place (such as a specific location on a biochip). If there is remanence on the microelectromagnetic dispenser head or the solidified, sample-magnetic bead complex 6 sticks to the microelectromagnetic dispenser head, the active dispensation methods can be used to dispense the sample-magnetic bead complex 6. FIG. 4 (B) shows one active dispensation method for dispensing the complex 6. The biochip 9 shown in FIG. 4 (B) is an active electromagnetic chip (see Chinese patent application 99104113.5, 99120320.8, “Individually addressable micro-electromagnetic unit array chips;” and WO 00/54882). This biochip has integrated electromagnetic units, which can generate magnetic fields in specific locations on the chip, such as a microelectromagnetic unit 10. When the microelectromagnetic dispenser head with sample-magnetic bead complex 6 is above the microelectromagnetic unit 10, the electrical signal (i.e. electrical current) in the microelectromagnetic dispenser is turned off to withdraw the magnetic field produced by the microelectromagnetic dispenser head and the electrical signal for microelectromagnetic unit 10 is turned on to generate a magnetic field; which attracts the sample-magnetic bead complex 6. FIG. 4 (C) shows another active dispensation method for sample-magnetic bead complex 6. This method can be used with any kind of biochip and sample container. An external magnet 11 is placed under the biochip or sample container, which will generate the magnetic field for attracting the sample-magnetic bead complex 6. After the solid sample-magnetic bead complex 6 is dispensed to target location, temperature is increased to melt the complex 6, which becomes magnetic bead 7 and liquid sample 8. The magnetic bead 7 can be removed by an external magnetic field. The microelectromagnetic dispenser head can be cleaned for next transportation and dispensation.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

What is claimed is:

1. A microelectromagnetic dispenser head, which head comprises:
a) a core comprising a magnetizable substance, said core surrounded by a microcoil suitable for transmitting electrical current and generating a magnetic field via said magnetizable substance and said core having a tip suitable for attracting a magnetic or magnetically labeled moiety; and
b) one or both of the following:
i) a shell that substantially shields magnetic field, generated via said microcoil, from the non-tip portion of said core; and/or
ii) a cooling means for cooling said tip.

2. The microelectromagnetic dispenser head of claim 1, wherein the core is in the shape of a cylinder, cube or cuboid.

3. The microelectromagnetic dispenser head of claim 1, wherein the magnetizable substance is selected from the group consisting of a paramagnetic substance, a ferromagnetic substance and a ferrimagentic substance.

4. The microelectromagnetic dispenser head of claim 1, wherein the magnetizable substance comprises a metal composition.

5. The microelectromagnetic dispenser head of claim 4, wherein the metal composition is a transition metal composition or an alloy thereof.

6. The microelectromagnetic dispenser head of claim 5, wherein the transition metal is selected from the group consisting of iron, nickel, copper, cobalt, manganese, tantalum, zirconium, nickel-iron alloy and cobalt-tantalum-zirconium (CoTaZr) alloy.

7. The microelectromagnetic dispenser head of claim 4, wherein the metal composition is Fe₃O₄.

8. The microelectromagnetic dispenser head of claim 1, wherein the core is surrounded by a single microcoil.

9. The microelectromagnetic dispenser head of claim 1, wherein the core is surrounded by a plurality of microcoils.

10. The microelectromagnetic dispenser head of claim 1, wherein the core is movably surrounded by a microcoil and the relative positional movement between the core and the microcoil can be used to adjust the generated magnetic field.

11. The microelectromagnetic dispenser head of claim 1, wherein the head comprises the core and the tip as an integral unit.

12. The microelectromagnetic dispenser head of claim 1, wherein the head comprises the core and the tip as separate units.

13. The microelectromagnetic dispenser head of claim 12, wherein the tip unit is replaceable.

14. The microelectromagnetic dispenser head of claim 1, wherein the core and the tip comprise the same or different magnetizable substance(s).

15. The microelectromagnetic dispenser head of claim 1, wherein the tip is in a sharp shape.

16. The microelectromagnetic dispenser head of claim 1, wherein the tip has a diameter at about 100, from 100 to about 50, about, from about 50 to about 20, about 20, from about 20 to about 10, about 10, from about 10 to about 5, about 5, from about 5 to about 2, about 2, from 2 to about 1, about 1, from about 1 to about 0.5, about 0.5 or less than 0.5 micron(s).

17. The microelectromagnetic dispenser head of claim 1, wherein the surface of the tip is convex, concave or plane.

18. The microelectromagnetic dispenser head of claim 1, wherein the surface of the tip is hydrophobic or hydrophilic.

19. The microelectromagnetic dispenser head of claim 1, wherein the surface of the tip is modified to comprise electrostatic charge.

20. The microelectromagnetic dispenser head of claim 1, wherein the shell comprises a high permeability material.

21. The microelectromagnetic dispenser head of claim 1, wherein the cooling means is a cooling material filled within the head.

22. The microelectromagnetic dispenser head of claim 1, wherein the cooling means is a high thermal conductivity material that is attached to the head and is in contact with the microcoil and in contact with an external low temperature source.

23. The microelectromagnetic dispenser head of claim 1, wherein the cooling means is a cooling device associated with or integrated within the head.

24. The microelectromagnetic dispenser head of claim 1, wherein the cooling means is attached to the head and is in contact with both ends of the microcoil.

25. The microelectromagnetic dispenser head of claim 1, which comprises a plurality of the tips.

26. The microelectromagnetic dispenser head of claim 25, wherein the distance among the tips corresponds to the distance among the magnetic or magnetically labeled moieties to be attracted.

27. The microelectromagnetic dispenser head of claim 1, which comprises both a shell that substantially shields magnetic field, generated via the microcoil, from the non-tip portion of the core, and a cooling means for cooling the tip.

28. An array of microelectromagnetic dispenser heads, which array comprises a plurality of the microelectromagnetic dispenser heads of claim 1.

29. The array of claim 28, wherein each of the microelectromagnetic dispenser head is individually addressable.

30. A microelectromagnetic dispenser, which dispenser comprises:
a) a microelectromagnetic dispenser head of claim 1; and
b) a means for controllably moving said head.

31. A microelectromagnetic dispenser, which dispenser comprises:
a) a microelectromagnetic dispenser head of claim 25; and
b) a means for controllably moving said head.

32. A microelectromagnetic dispenser, which dispenser comprises:
a) an array of microelectromagnetic dispenser heads of claim 28; and
b) a means for controllably moving said array.

33. The microelectromagnetic dispenser of claim 32, wherein each of the microelectromagnetic dispenser head is individually addressable.

34. A method for transferring a moiety, which method comprises:
a) providing a magnetic or magnetically labeled moiety to be transferred at first location,
b) providing a microelectromagnetic dispenser head comprising a core comprising a magnetizable substance, said core surrounded by a microcoil suitable for transmitting electrical current and generating a magnetic field via said magnetizable substance and said core having a tip suitable for attracting said magnetic or magnetically labeled moiety;
c) attracting said magnetic or magnetically labeled moiety from said first location to said tip of said microelectromagnetic dispenser head; and
d) transferring and releasing said magnetic or magnetically labeled moiety to a second location from said tip of said microelectromagnetic dispenser head.

35. The method of claim 34, wherein the magnetic or magnetically labeled moiety is a magnetized cell, cellular organelle, virus, molecule and an aggregate or complex thereof.

36. The method of claim 34, wherein the magnetically labeled moiety is provided by attaching a moiety to a magnetic particle.

37. The method of claim 36, wherein the magnetic particle comprises a binding partner that is capable of binding to a moiety to be transferred.

38. The method of claim 34, wherein the binding partner is capable of specifically binding to a moiety to be transferred.

39. The method of claim 34, wherein the binding partner is an antibody or a nucleotide sequence.

40. The method of claim 34, wherein the binding partner is selected from the group consisting of a cell, cellular organelle, virus, molecule and an aggregate or complex thereof.

41. The method of claim 34, wherein the magnetic particle comprises a plurality of binding partners, each binding partner is capable of binding or specifically binding to a different moiety.

42. The method of claim 34, wherein the microelectromagnetic dispenser head further comprises a shell that substantially shields magnetic field, generated via the microcoil, from the non-tip portion of the core of the microelectromagnetic dispenser head.

43. The method of claim 34, wherein the microelectromagnetic dispenser head is part of a microelectromagnetic dispenser that further comprises a means for controllably moving the head.

44. The method of claim 34, wherein the magnetic field in the attracting, transferring and/or releasing step(s) is adjusted.

45. The method of claim 44, wherein the magnetic field is adjusted by a relative positional movement between the core and the microcoil of the microelectromagnetic dispenser head.

46. The method of claim 44, wherein the magnetic field is adjusted by adjusting electric current in the microcoil.

47. The method of claim 44, wherein the magnetic field is adjusted by an external augmentative or counter magnetic field.

48. The method of claim 47, wherein the external augmentative or counter magnetic field is effected via an external magnet or electromagnetic unit.

49. The method of claim 34, wherein the magnetic or magnetically labeled moiety is released by shutting off the electric current in the microcoil.

50. The method of claim 34, wherein the magnetic or magnetically labeled moiety in the attracting, transferring and/or releasing step(s) is cooled.

51. The method of claim 50, wherein the cooling is effected via a cooling material filled within the head or a cooling device associated with or integrated within the head of the microelectromagnetic dispenser.

52. The method of claim 50, wherein the magnetic or magnetically labeled moiety at the first location is in a liquid state and is cooled to be in a solid state in the attracting, transferring and/or releasing step(s).

53. The method of claim 50, further comprising a heating step to facilitate attracting, transferring and/or releasing of the magnetic or magnetically labeled moiety, said heating step not changing the magnetic or magnetically labeled moiety from solid state to liquid state.

54. The method of claim 34, wherein all magnetic or magnetically labeled moieties are attracted, transferred and/or released from a first location to a second location.

55. The method of claim 34, further comprising identifying the magnetic or magnetically labeled moieties containing a non-magnetic, identifiable signal and attracting, transferring and/or releasing such identified magnetic or magnetically labeled moieties from a first location to a second location.

56. The method of claim 55, wherein the non-magnetic, identifiable signal is an optical signal.

57. The method of claim 36, wherein the magnetic particle comprises an optical labeling substance.

58. The method of claim 34, wherein the first and/or second location is selected from the group consisting of a beaker, a flask, a cylinder, a test tube, an enpindorf tube, a centrifugation tube, a culture dish, a multiwell plate, a filter membrane, a microscopic slide and a chip.

59. The method of claim 34, wherein the microelectromagnetic dispenser head comprises a plurality of the tips and a plurality of the magnetic or magnetically labeled moieties are attracted, transferred and/or released from a first plurality of locations to a second plurality of locations.

60. The method of claim 34, wherein an array of the microelectromagnetic dispenser heads are used and a plurality of the magnetic or magnetically labeled moieties are attracted, transferred and/or released from a first plurality of locations to a second plurality of locations.

61. The method of claim 34, wherein a magnetically labeled moiety is transferred from a first location to a second location and further comprising recovering said transferred moiety from said magnetic label.
